(19) United States
(12) Patent Application Publication
(10) Pub. No.: US 2026/0202393 A1
(43) Pub. Date: Jul. 16, 2026

(54) HYPERSONIC OXIDIZATION THERMAL TESTING WITH EMPIRICAL RESEARCH

(71) Applicant: CFD Research Corporation, Huntsville, AL (US)

(72) Inventor: Joseph Roger Herdy, JR., Huntsville, AL (US)

(21) Appl. No.: 19/562,476

(22) Filed: Mar. 10, 2026

Related U.S. Application Data
(63) Continuation-in-part of application No. 17/502,527, filed on Oct. 15, 2021.
(60) Provisional application No. 63/092,280, filed on Oct. 15, 2020.
(60) Provisional application No. 63/770,851, filed on Mar. 12, 2025.

(51) Int. Cl. G01N 33/208

(52) U.S. Cl. CPC G01N 33/208

(57) ABSTRACT

A method of controlling nitrogen and oxygen content of synthetic air is disclosed. The method may include decomposing nitrous in a decomposition system by expanding liquid nitrous into gaseous nitrous in a decomposition chamber. A gaseous mixture of oxygen gas (O2), transient oxygen atoms (O), nitrogen gas (N2), and/or transient nitrogen atoms (N) may be obtained from the decomposition. The gaseous mixture may be provided to a test chamber that is at least initially devoid of receiving an additional nitrogen gas (N2) from either an N2 supply or a reservoir of air and/or at least initially devoid of receiving additional oxygen gas (O2) from the reservoir of air. The test material may be exposed to the gaseous mixture in the test chamber. A response of the test material to the gaseous mixture may be determined.

### CROSS-REFERENCE TO RELATED APPLICATIONS

This patent application claims priority to and the benefit of U.S. Provisional Patent Application Ser. No. 63/770,851, entitled HYPERSONIC OXIDIZATION THERMAL TESTING WITH EMPIRICAL RESEARCH, filed Mar. 12, 2025, which is incorporated by reference in its entirety.

This patent application is also a continuation in part of U.S. application Ser. No. 17/502,527 entitled “NITROUS DECOMPOSITION WITHOUT CATALYST” filed on Oct. 15, 2021, which claims priority to and the benefit of U.S. Provisional Patent Application Ser. No. 63/092,280 filed Oct. 15, 2020, which are incorporated by reference in their entirety.

### U.S. GOVERNMENT RIGHTS

This invention was made with government support under AMTC-SBIR-23-07-001 awarded by the U.S. Army Aviation and Missile Technology Consortium (AMTC). The government has certain rights in the invention.

### BACKGROUND

Previously, it has been difficult to simulate flight conditions in relevant environments in ground test configurations, such as sustained cruise at high Mach numbers, and obtain relevant flight data for real world flight conditions. Additionally, it has also been difficult to obtain realistic simulated flight condition data for simulating the physical effects from extra oxygen which may duplicate or simulate an accelerated oxygen interaction environment. Currently, it is difficult to study phenomena associated with oxygen, such as ionization of air, which may be representative of vehicle flight re-entry conditions. Moreover, it has been difficult to obtain realistic flight data for simulations while modulating flow characteristics, such as modulating amounts of different constituents of air (e.g., primarily the abundant substances found naturally in air, such as nitrogen and oxygen), in ground tests over mission duration time scales.

The subject matter claimed herein is not limited to embodiments that solve any disadvantages or that operate only in environments such as those described above. Rather, this background is only provided to illustrate one example technology area where some embodiments described herein may be practiced.

### SUMMARY

In some embodiments, the systems and methods described herein allow for modulating flow characteristics and substance content during ground testing, such as in a wind tunnel. The systems and/or methods described herein allow for creation of high enthalpy hypersonic synthetic air conditions via decomposition of nitrous oxide into pure oxygen atoms and nitrogen atoms and their gaseous molecular embodiments (e.g., gaseous oxygen (O₂) and gaseous nitrogen (N₂)) at relative and realistic pressures and temperatures, such as at high Mach. This process may be performed with or without adding additional nitrogen gas, oxygen gas, or other gases. As such, during the operation of the wind tunnel, the inlet gas flow characteristics may be modulated to obtain variations in the gas with relation to the decomposition of nitrous oxide into oxygen atoms and nitrogen atoms as well as the molecular oxygen gas and molecular nitrogen gas (both diatomic molecules as in the Earth's atmosphere). However, it should be recognized that some recombination of atomic nitrogen and atomic oxygen may occur to form O₂, N₂, NO₂, and NO, as well as others. The test conditions may determine the atomic species duration and any recombination. It should be recognized that any atomic species may be present at a duration, which is dependent on the conditions. As such, the atomic oxygen and atomic nitrogen may be considered to be intermediate oxygen atoms and intermediate nitrogen atoms, until they are involved in a reaction to obtain a molecular species.

In some embodiments, the generation of gases may be obtained by operation of the system described in U.S. Pat. App. Pub. No. 2022/0119256 (U.S. application Ser. No. 17/502,527) entitled “NITROUS DECOMPOSITION WITHOUT CATALYST”, which is incorporated herein by specific reference in its entirety. Now, the system may be used to develop molecular species that are naturally present at interactions of high Mach and/or hypersonic conditions in flight such that air experienced during hypersonic flight conditions may be simulated. Thereby, hypersonic ground testing may be enabled as well as correlation of real-world flight data and simulated flight data.

In some embodiments, the system and/or methods described herein may restrict any addition of nitrogen into the system before or after the decomposition of nitrous oxide into oxygen and nitrogen atoms. In some instances, additional nitrogen into the nitrous oxide decomposition flow may be inhibited or reduced at certain predetermined times and/or rates. For example, the reduction or elimination of additional nitrogen gas into the nitrous oxide decomposition system may be performed to induce higher oxygen concentrations above normal atmospheric composition by reducing the amount of nitrogen available such that thermal protection systems (TPS) may be tested at higher oxygen concentrations. This results in less than atmospheric nitrogen concentration and higher than atmospheric oxygen concentration. The decrease in nitrogen concentration and increase in concentration of oxygen may be used to accelerate and/or parameterize oxygen effects (e.g., oxidation) on different test materials or propulsion systems used in flight environments.

In some embodiments, the system may implement spontaneous generated enthalpy of the decomposition of nitrous oxide, with or without the addition of nitrogen. The nitrogen may be initially included and then turned off to increase oxygen content and simulate oxidizing conditions. In some instances, the system may omit introduction of additional nitrogen by inhibiting the additional nitrogen gas inlet.

In some embodiments, the system may perform the nitrous decomposition with added or subsequent extra nitrogen to cause the nitrogen concentration to be higher than normal air, which results in lower oxygen concentration than normal air.

In some embodiments, a method of controlling nitrogen and oxygen content of synthetic air may include: decomposing nitrous in a decomposition system by expanding liquid nitrous into gaseous nitrous in a decomposition chamber of the decomposition system, wherein the gaseous nitrous obtains a temperature for decomposition; obtaining a gaseous mixture of oxygen gas (O₂), intermediate oxygen atoms (O), nitrogen gas (N₂), and/or intermediate nitrogen atoms (N) from the decomposition, wherein the oxygen atom to nitrogen atom ratio is about 1:2 (oxygen atoms/molar: nitrogen atoms/molar); and providing the gaseous mixture to a test chamber, wherein the test chamber may be at least initially devoid of receiving an additional nitrogen gas (N₂) and/or at least initially devoid of receiving additional oxygen gas (O₂); exposing the test material to the gaseous mixture in the test chamber; and determining a response of the test material to the gaseous mixture. The oxygen atoms and nitrogen atoms may be intermediates that react and may also be considered to be transient until forming a molecular species.

In some embodiments, a method of controlling nitrogen and oxygen content of synthetic air may include: decomposing nitrous in a decomposition system by expanding liquid nitrous into gaseous nitrous in a decomposition chamber of the decomposition system, wherein the gaseous nitrous obtains a temperature for decomposition; obtaining a gaseous mixture of oxygen gas (O₂), transient oxygen atoms (O), nitrogen gas (N₂), and/or transient nitrogen atoms (N) from the decomposition, wherein the oxygen atom to nitrogen atom ratio is about 1:2 (oxygen atoms/molar: nitrogen atoms/molar); providing the gaseous mixture to a test chamber; providing an additional nitrogen gas (N₂) into the test chamber to obtain a nitrogenated gaseous mixture, wherein the test chamber is optionally receiving an additional oxygen gas (O₂), wherein the nitrogenated gaseous mixture has a nitrogen content of either: less than or about 77% nitrogen gas (N₂) and transient nitrogen atoms (N) by volume and greater than 22% oxygen gas (O₂) and transient oxygen atoms (O) by volume; or greater than 80% nitrogen gas nitrogen gas (N₂) and transient nitrogen atoms (N) by volume and less than 21% oxygen gas (O₂) and transient oxygen atoms (O) by volume; exposing a test material to the nitrogenated gaseous mixture in the test chamber; and determining a response of the test material to the nitrogenated gaseous mixture.

In some embodiments, a method of controlling nitrogen and oxygen content of synthetic air may include: decomposing nitrous in a decomposition system by expanding liquid nitrous into gaseous nitrous in a decomposition chamber of the decomposition system, wherein the gaseous nitrous obtains a temperature for decomposition; obtaining a gaseous mixture of oxygen gas (O₂), transient oxygen atoms (O), nitrogen gas (N₂), and/or transient nitrogen atoms (N) from the decomposition, wherein the oxygen atom to nitrogen atom ratio is about 1:2 (oxygen atoms/molar: nitrogen atoms/molar); providing the gaseous mixture to a test chamber; providing an additional nitrogen gas (N₂) into the test chamber to obtain a nitrogenated gaseous mixture, wherein the test chamber is devoid of receiving an additional oxygen gas (O₂) input, wherein the nitrogenated gaseous mixture has a nitrogen content of either: less than or about 77% nitrogen gas (N₂) and transient nitrogen atoms (N) by volume and greater than 22% oxygen gas (O₂) and transient oxygen atoms (O) by volume; or greater than 80% nitrogen gas nitrogen gas (N₂) and transient nitrogen atoms (N) by volume and less than 21% oxygen gas (O₂) and transient oxygen atoms (O) by volume; exposing a test material to the nitrogenated gaseous mixture in the test chamber; and determining a response of the test material to the nitrogenated gaseous mixture.

In some embodiments, the methods may also include using pressure sensitive paint, which functions by emitting photons (i.e. luminesce), under illumination of lamps or diodes of a specific wavelength, which are used to excite a pressure sensitive probe within the paint. Once excited, the pressure sensitive probe is transitioned to a higher energy state where it may either emit a photon or be quenched by local oxygen present. This competing process of emission and quenching determines the intensity response of the paint layer. The result is a dimmer fluorescence where there is higher pressure and brighter response at lower pressures, thus providing a visual mapping correlating to the pressure environment and the time exposed. Tailoring nitrogen and oxygen ratios of the synthetic air in combination with the use of pressure sensitive paint may allow for mapping pressure contours of the test material based on the response of the pressure sensitive paint to nitrogenated air having more nitrogen content than normal air and less oxygen content than normal air. In some embodiments, a pressure sensitive paint may be applied to a test surface of the test material before exposing the test material to the gaseous mixture and the method may further include: measuring brightness of luminescence of the pressure sensitive paint in response to the test material being exposed to the gaseous mixture; determining areas of high brightness to have low oxygen concentration or low oxygen pressure; determining areas of low brightness to have high oxygen concentration or high oxygen pressure; and mapping a continuous pressure field of the test surface based on the measured brightness. In some instances, the gaseous mixture may have a nitrogen concentration that is higher than ambient air and an oxygen concentration of less than 21% by volume or moles when the test material is exposed to the gaseous mixture.

Both the foregoing general description and the following detailed description are given as examples and are explanatory and are not restrictive of the invention, as claimed.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1 illustrates a wind tunnel system that may be used in connection with controlling the nitrogen and/or oxygen content of synthetic air;

FIG. 2 illustrates a pressure sensitive paint (PSP) measurement diagram;

FIG. 3 illustrates an example testing apparatus and a testing paradigm of a hypersonic vehicles' scramjet engine; and

FIG. 4 illustrates data from a test in a hypersonic research tunnel known as the Hypersonic Synthetic Environment Test Tunnel (HySETT);

FIG. 5 illustrates example conditions at altitude that may be experienced by a hypersonic vehicle during flight, with some of the regions of atomic dissociation noted; and

FIG. 6 illustrates an example computing device that may be used in the systems and methods described throughout this disclosure.

The elements and components in the figures may be arranged in accordance with at least one of the embodiments described herein, and which arrangement may be modified in accordance with the disclosure provided herein by one of ordinary skill in the art.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, may be arranged, substituted, combined, separated, and designed in a wide variety of different configurations, all of which are explicitly contemplated herein.

In some embodiments, the systems and/or methods described may also be described as Hypersonic Oxidization Thermal Testing with Empirical Research (HOTTER). A method of HOTTER may be performed and may include controlling nitrogen and/or oxygen content of synthetic air. The method may include performing nitrous decomposition in a decomposition system by expanding liquid nitrous into gaseous nitrous in a decomposition chamber. A gaseous mixture of oxygen gas, transient oxygen atoms, nitrogen gas, and/or transient nitrogen atoms may be obtained from the decomposition. The gaseous mixture may be provided to a test chamber. The test chamber may be at least initially devoid of receiving additional nitrogen gas and/or oxygen gas from an external supply. A test material in the test chamber may be exposed to the gaseous mixture. A response of the test material to the gaseous mixture may be determined.

A method of controlling nitrogen and oxygen content of synthetic air may include decomposing nitrous in a decomposition system by expanding liquid nitrous into gaseous nitrous in a decomposition chamber, wherein the gaseous nitrous may obtain a temperature for decomposition. A gaseous mixture of oxygen gas, transient oxygen atoms, nitrogen gas, and/or transient nitrogen atoms may be obtained from the decomposition, wherein the oxygen atom to nitrogen atom ratio may be about 1:2 (oxygen atoms/molar: nitrogen atoms/molar). The gaseous mixture may be provided to a test chamber, wherein the test chamber may be at least initially devoid of receiving additional nitrogen gas and/or additional oxygen gas from either a N₂ supply or a reservoir of air. A test material in the test chamber may be exposed to the gaseous mixture. A response of the test material to the gaseous mixture may be determined.

A method of controlling nitrogen and oxygen content of synthetic air may include decomposing nitrous in a decomposition system by expanding liquid nitrous into gaseous nitrous in a decomposition chamber, wherein the gaseous nitrous may obtain a temperature for decomposition. A gaseous mixture of oxygen gas, transient oxygen atoms, nitrogen gas, and/or transient nitrogen atoms may be obtained from the decomposition, wherein the oxygen to nitrogen ratio may be about 1:2 (oxygen atoms/molar: nitrogen atoms/molar). The gaseous mixture may be provided to a test chamber, wherein the test chamber may be at least initially devoid of receiving additional nitrogen gas. A test material may be exposed to the gaseous mixture in the test chamber. A response of the test material to the gaseous mixture may be determined.

A method of controlling nitrogen and oxygen content of synthetic air may include decomposing nitrous in a decomposition system by conditioning liquid nitrous into gaseous nitrous in a decomposition chamber, wherein the gaseous nitrous may obtain a temperature for decomposition. A gaseous mixture of oxygen gas, transient oxygen atoms, nitrogen gas, and/or transient nitrogen atoms may be obtained from the decomposition, wherein the oxygen atom to nitrogen ratio may be about 1:2 (oxygen atoms/molar: nitrogen atoms/molar). The gaseous mixture may be provided to a test chamber, wherein the test chamber may be at least initially devoid of receiving additional nitrogen gas or transient nitrogen atoms input. Additional oxygen gas and/or transient oxygen atoms input may be provided into the test chamber. A test material or system may be exposed to the gaseous mixture in the test chamber. A response of the test material to the gaseous mixture may be determined.

A method of controlling nitrogen and oxygen content of synthetic air may include decomposing nitrous in a decomposition system by conditioning liquid nitrous into gaseous nitrous in a decomposition chamber, wherein the gaseous nitrous may obtain a temperature for decomposition. A gaseous mixture of oxygen gas, transient oxygen atoms, nitrogen gas, and/or transient nitrogen atoms may be obtained from the decomposition, wherein the oxygen atom to nitrogen atom ratio may be about 1:2 (oxygen atoms/molar: nitrogen atoms/molar). The gaseous mixture may be provided to a test chamber. Additional nitrogen gas may be provided into the test chamber to obtain a nitrogenated gaseous mixture, wherein the test chamber may be at least initially devoid of receiving additional oxygen gas input. The nitrogenated gaseous mixture may have a nitrogen content of either: less than or about 77% nitrogen gas and transient nitrogen atoms by volume or moles and greater than 22% oxygen gas and transient oxygen atoms by volume or moles; or greater than 80% nitrogen gas and transient nitrogen atoms by volume or moles and less than 21% oxygen gas and transient oxygen atoms by volume or moles. A test material may be exposed to the nitrogenated gaseous mixture in the test chamber. A response of the test material to the nitrogenated gaseous mixture may be determined.

A method of controlling nitrogen and oxygen content of synthetic air may include decomposing nitrous in a decomposition system by expanding liquid nitrous into gaseous nitrous in a decomposition chamber, wherein the gaseous nitrous may obtain a temperature for decomposition. A gaseous mixture of oxygen gas, transient oxygen atoms, nitrogen gas, and/or transient nitrogen atoms may be obtained from the decomposition, wherein the oxygen atom to nitrogen atom ratio may be about 1:2 (oxygen atoms/molar: nitrogen atoms/molar). The gaseous mixture may be provided to a test chamber. Additional nitrogen gas may be provided into the test chamber to obtain a nitrogenated gaseous mixture wherein the test chamber may be optionally receiving additional oxygen gas input. The nitrogenated gaseous mixture may have a nitrogen content of either: less than or about 77% nitrogen gas and transient nitrogen atoms by volume or moles and greater than 22% oxygen gas and transient oxygen atoms by volume or moles; or greater than 80% nitrogen gas and transient nitrogen atoms by volume or moles and less than 21% oxygen gas and transient oxygen atoms by volume or moles. A test material may be exposed to the nitrogenated gaseous mixture in the test chamber. A response of the test material to the nitrogenated gaseous mixture may be determined.

In some embodiments, the systems and/or methods may be used to ground test different features of flight vehicles and/or the associated propulsion systems in a wind tunnel environment by restricting or omitting additional nitrogen gas introduction into the flow path of the synthetic air. Accordingly, the system may implement a process to expose materials used in conjunction with high speed/high Mach and above (i.e., hypersonic conditions) for flight vehicle or ground test components that may benefit from empirical research of their exposure to elevated oxygen levels (e.g., relative to nominal atmospheric conditions) in some embodiments, or elevated nitrogen levels in some embodiments. The data obtained from this testing provides insights into oxygen impacts on these materials from oxygen and/or nitrogen levels that are not normally found in nature. The data may be used to derive functionality, compatibility, reliability, sustainability, and/or longevity for components of flight vehicles, such as thermal protection system (TPS) components, structures that compose vehicle structures, and/or interior flow path surfaces of scramjet engines. The test conditions may be obtained by reducing or eliminating extra nitrogen from being introduced into the system. That is, in some embodiments, no additional nitrogen is introduced over the nitrogen that is obtained by decomposition of the nitrous oxide, which provides an oxygen enriched, thermally conditioned gas. The thermally conditioned oxygenated gas is created as an adjunct option when creating hypersonic conditions for testing as is described in U.S. Pat. App. Pub. 2022/0119256 A1 (U.S. application Ser. No. 17/502,527) entitled “NITROUS DECOMPOSITION WITHOUT A CATALYST,” which is incorporated herein in its entirety by specific reference. In some aspects, the system is operated where the addition of extra nitrogen gas is curtailed, forgone, inhibited, or otherwise modulated to create a desired environment of oxygen enriched “air” via decomposition of nitrous oxide. That is, active control of the additional nitrogen supply may be performed, whether manual or controlled by the process control system (e.g., controller, such as a computer system) of the nitrous decomposition systems described herein. However, adding extra nitrogen to have reduced oxygen may be useful in some testing, such as with pressure sensitive paints.

Accordingly, the inhibition of nitrogen addition provides the ability to simulate flight conditions in relevant environments (e.g., such as sustained cruise at high Mach numbers, or even rarified ionization of air duplicating shorter periods of vehicle re-entry conditions), while modulating flow characteristics, such as oxygen content in ground tests over mission duration time scales. Accordingly, the systems and nitrogen restricting protocols may be coupled with modeling and simulation on the computing system and may be used for modeling different materials for TPS. For example, internal and external structures of a flight vehicle that may be studied for impact of oxygen content may include any vehicle surface, such as a vehicle bow, ogive surfaces, and/or air breathing engine inlet openings (e.g., either protruding and/or submerged), and/or control surfaces (e.g., composite structures). The restriction of nitrogen may be used in predicting effects from oxidation that may be present on different components and associated surfaces.

In some aspects, the restriction of nitrogen may be used for predicting oxidation or combustion effects that are tied to heat flux with accuracy. The considerations may also include complex multi-physics phenomena typical of hypersonic flows, in particular gas-surface interactions. For example, determining the response of the test material may include determining a degree of oxidation of the test material resulting from combustion heat flux. In these and other embodiments, an oxidation model of the test material may be generated based on the response of the test material. In these and other embodiments, a predicted degree of oxidation resulting from combustion heat flux under predetermined conditions may be determined based on the oxidation model.

In some embodiments, the restriction of nitrogen may be supplemented by increasing introduction of oxygen gas or any oxygen-generating precursor that does not also generate nitrogen. The technique for varying the oxygen content and the exposure to materials may be applied to other applications, such as the accelerated “aging” of materials and fuels used in solid rocket motors, which may provide predictive capabilities not practical from conventional surveillance techniques.

In some embodiments, decomposing the nitrous may include conditioning liquid nitrous into gaseous nitrous in the decomposition chamber. In some embodiments, heated nitrogen gas may be injected into the decomposition chamber to mix with and/or modulate the gaseous nitrous and the injection of the heated nitrogen gas may be regulated to obtain the temperature for decomposition of the gaseous nitrous. For example, the gaseous nitrous may be heated to the temperature for decomposition at least partially by the injection of the heated nitrogen gas. In these and other embodiments, the temperature of decomposition may be at least about 900 degrees K. In some embodiments, the gaseous nitrous may be decomposed into the gaseous mixture, and the gaseous mixture may include the nitrogen gas and the oxygen gas. In some embodiments, the injection of the heated nitrogen gas may be restricted to increase a concentration of the oxygen gas in the gaseous mixture. In these and other embodiments, the test material in the test chamber may be analyzed based on the increased concentration of oxygen gas.

In some embodiments, the heated nitrogen gas may be injected into the decomposition chamber for a first period of time. The injection of the heated nitrogen gas may be restricted or terminated for a second period of time after the first period of time. In some embodiments, the heated nitrogen gas may be optionally reinjected into the decomposition chamber for a third period of time after the second period of time. In these and other embodiments, the oxidation behavior of the test material may be determined during the first period of time and/or second period of time and/or the third period of time.

In some embodiments, the test material may be part of a TPS of a vehicle, such as a plane, missile, rocket, projectile, carrier, flying object, or drone. In some embodiments, the test material may include an engine and the methods described herein may optionally include performing an engine combustion test. In some embodiments, the test material may include rocket material. In some embodiments, the test material may include one or more composites such as a SiC composite, a ZrBR₂ composite, a ZrB₂ composite, a ZrB₂—SiC composite, a ZrB₂—ZrC—SiC composite. In some embodiments, the test material may include a refractory alloy and/or a ceramic. In some embodiments, the test material may include at least one of a composite, a refractory alloy, and/or a ceramic.

For example, the test material may include SiC based materials for TPS components subject to temperatures <1600° C. At this temperature range, though active oxidation still emerges at quite low initial pressure Po (ambient pressure), a continuous SiO₂ layer may still survive and thus protect the material from further oxidation. Beyond 1600° C. some SiC based TPS may still be ablation resistant, especially in on-ground simulated environments. For example, when measured inside an inductively coupled plasma (ICP) wind tunnel, the traditional carbon fiber reinforced SiC matrix composite (e.g., Cf/SiC) may survive up to ~1750° C., beyond which the surface of the SiC coating of Cf/SiC dissipates, leading to an evident ‘temperature jump’ and failure of the whole material. The ‘temperature jump’ is a main phenomenon of SiC ablation. It emerges in almost all SiC based composites when served in re-entry environments.

The ‘temperature jump’ mechanisms have been well studied previously at ground test facilities such as the von Karman Institute for Fluid Dynamics (VKI). It is now accepted that the ‘temperature jump’ of SiC is triggered by the active oxidation and recombination of atomic species (e.g., mainly O and N). Due to the exothermic reactions of both processes, the thermal balance achieved prior to the ‘temperature jump’ is broken, thus leading to an abrupt temperature increase until another balance is achieved at a higher temperature level (generally >2000° C.). This may now be tested in the nitrogen deficient environments described herein.

In some embodiments, the anticipated lower threshold of active oxidation may be achieved with higher oxygen content and less nitrogen content for TPS materials, such as SiC. This occurs with the utilization of higher than naturally occurring atomic oxygen levels created in the hypersonic conditions, which is inferred by the simulated flight-like condition recovery temperatures and pressures associated with materials used in the experimental testing of the hypersonic tunnel utilizing decomposed nitrous oxide.

In some embodiments, the system may be used for testing the oxidation behavior of composites, such as ZrB₂-based composites, under ambient pressure, and significant progress has been made in the testing of these composites. However, very little attention has been paid to the oxidation of the ZrB₂-based composites in atomic oxygen in simulated flight conditions. As well known, during hypersonic flight in the atmosphere, a large amount of oxygen molecules in the bow shock are dissociated into atoms, which partially diffuse to the vehicle surface through the boundary layer. Then, these oxygen atoms on the surface will recombine into molecules accompanied by the oxidation of wall material. This degrades the wall by removing atoms that are oxidized. The oxidation behavior of, for example, ZrB₂—SiC composites under high enthalpy, low pressure and atomic oxygen conditions is significantly different from that under ambient pressure. Researchers have demonstrated the formation and evolution of zircon during the oxidation of ZrB₂—SiC composites at 200 Pa and found that the main reason for the formation of zircon may be attributed to the active oxidation of SiC. Researchers have also investigated the oxidation behavior of ZrB₂—SiC composites at 1800° C. with oxygen partial pressures of 0.2 and 2×10⁴ atm, respectively. The effects of partial pressure on the microstructure of oxide scale indicated that low oxygen partial pressure was detrimental to the oxidation resistance of the composites because of the active oxidation of SiC. Nevertheless, the quantitative analysis of the oxidation for ZrB₂—SiC composites in a wide range of oxygen partial pressure has not been explored. Now, the oxygen rich environment created with the systems and/or methods described herein may be used to study these materials with higher relevancy.

Oxygen atoms may also have a significant influence on TPS materials. The degradation of Ni-based alloys and carbon-based materials in dissociated and molecular oxygen has been observed. The experimental results showed that these materials exhibited high degradation in atomic oxygen. Researchers investigated the oxidation of SiC under simulated atmospheric re-entry conditions. Atomic oxygen significantly affected the oxidation kinetics as well as the crystalline phase of resulting SiO₂ and may accelerate the volatilization of silica. However, the transition between active and passive oxidation was not influenced by atomic oxygen. On the contrary, other researchers have pointed out the passive oxidation domain was significantly enlarged in atomic oxygen. The understanding of the response of ZrB₂-based composites to atomic oxygen lagged behind that of conventional TPS materials. Now, however, the response of ZrB₂-based composites to atomic oxygen may be monitored by reducing, restricting, or terminating additional nitrogen, so that the oxygen content increases without adding additional oxygen gas. Thus, the systems and methods may be operated to intentionally introduce and/or modulate higher oxidation levels on TPS materials to empirically study the effects by only reducing or eliminating additional nitrogen without adding additional oxygen gas.

The effect of atomic oxygen adsorption on the oxidation behavior of ZrB₂—ZrC—SiC composites in air has also been reported by researchers, indicating that the atomic oxygen adsorption on the surface enhanced the oxidation of the composites. However, the oxidation mechanism of ZrB₂-based composites has not been studied sufficiently. Thus, the use of these composites in hypersonic vehicles requires a comprehensive understanding of the oxidation behavior, especially under simulated re-entry conditions.

In some embodiments, the system and methods described herein may be used to apply higher than natural oxidation levels by design on composite structures, resulting in “artificial or simulated aging” effects on composites.

In some embodiments, the systems and methods as described herein have the ability to artificially accelerate and/or modulate the oxygen absorption onto and within composite structures, thereby offering an empirical environment for studying material response. This may be achieved by restricting or terminating additional nitrogen supply into the flow stream.

Additionally or alternatively, the systems and methods may be used in investigations to improve the fundamental understanding, modeling, and simulation efforts of the aging behavior of materials, such as ammonium perchlorate (AP)/hydroxyl-terminated polybutadiene (HTPB)/aluminized (Al) propellants. In some aspects, the restriction of nitrogen and associated increase in oxygen content may be used to identify aging processes that occur on a microscale and affect propellant safety and performance. This allows the restricted nitrogen protocol that is performed to increase oxygen content used in monitoring the aging processes for oxidative thermal aging of rocket fuel material including solid rocket motor fuel, such as crosslinked hydroxyl-terminated polybutadiene (HTPB)/isophorone diisocyanate (IPDI) polyurethane rubber. The system allows for the fuel to be studied at temperatures outside of traditional temperatures between 25° C. and 125° C. For example, changes in tensile elongation, mechanical hardening, polymer network properties, density, O₂, permeation and/or molecular chain dynamics may be investigated as a function of age or as a function of increased oxygen exposure. The increased oxygen exposure may be correlated with the increased aging of the rocket fuel material. The techniques that may be used include solvent swelling, detailed modulus profiling, and/or nuclear magnetic resonance relaxation measurements. The Arrhenius methodology, which normally assumes a linear extrapolation of high temperature aging data, may be evaluated by using extensive data superposition and highly sensitive oxygen consumption measurements. Significant curvature in the Arrhenius diagram of these oxidation rates was observed to be similar to previous results found for other rubber materials that have been evaluated by this technique. Studies of gel/network properties suggest that crosslinking is the dominant process at higher temperatures. The effect on the oxidation rate of the binder when other constituents found in propellants are present, such as ammonium perchlorate, plasticizer and aluminum powder, may also be determined.

In some embodiments, the systems and methods may be used to intentionally introduce and/or modulate higher oxidation levels on rocket fuel materials at modulated temperatures to empirically study the effects on the fuel. A potential for assessment of the fuel performance could also be possible via the active ignition and performance of said fuels in a tunnel providing the flow conditions to simulate the combustion environment, and this embodiment could also be applied to hybrid solid rocket motor studies.

In some embodiments, a pressure sensitive paint may be applied to a surface of a test material and may be used in connection with the methods described herein, such as in a wind tunnel. The system may be configured to vary the oxygen to nitrogen ratio as described herein, such as by increasing nitrogen greater than atmospheric concentrations and reducing oxygen less than atmospheric concentrations. In some aspects, the methods may be operated to intentionally introduce extra nitrogen, yield an atmosphere of lower than 21% oxygen by volume or moles, and/or use the generated synthetic air on pressure sensitive paints on test surfaces of test materials in the wind tunnel.

The pressure sensitive paint is based on the oxygen-quenching phenomenon of luminescence of specific organic luminophores. For a given excitation level, the brightness of the luminescent material varies inversely with the partial pressure of oxygen and, hence, to the pressure of the air. When the pressure sensitive paint is applied to a test surface (e.g., an aerodynamic surface) of a test material and placed in an operating wind tunnel under appropriate lighting, the molecules of the pressure sensitive paint luminesce as a function of the local pressure of oxygen over the surface of interest during aerodynamic flow. The resulting image obtained by a camera or other imaging device will be brightest in the areas of low pressure (e.g., areas of low oxygen concentration), and less intense in the areas of high pressure (e.g., areas of high oxygen concentration). The luminescence data may be used to map a continuous pressure field of the test surface of the test material based on the measured brightness. There will be a difference in the way the pressure sensitive paint may map flow fields when the nitrogen levels are varied. Accordingly, the testing may start at one nitrogen level for the synthetic air and then be changed to a second nitrogen level for the synthetic air. The change may be gradual so that a gradient of nitrogen concentrations may be used for assessing the response of the pressure sensitive paint.

As an example, a pressure sensitive paint may be applied to a test surface of the test material before exposing the test material to the gaseous mixtures described throughout the disclosure (e.g., the gaseous mixtures, nitrogenated gaseous mixtures, and/or oxygenated gaseous mixtures). The brightness of luminescence of the pressure sensitive paint may be measured in response to the test material being exposed to the gaseous mixture. Areas of high brightness may be determined to have low oxygen concentration and/or low oxygen pressure. Areas of low brightness may be determined to have high oxygen concentration or high oxygen pressure. In these and other embodiments, the measured brightness may be used to map a pressure field corresponding to the test surface based on the measured brightness. The pressure field may be used to determine locations on the test material that may be subject to higher oxidation rates, which may be used to modify the design of test materials, particularly with respect to TPS components. In some embodiments, the gaseous mixture to which the test surface may be exposed may have a nitrogen concentration higher than ambient air and an oxygen concentration of less than 21% by volume or moles.

In some aspects, the pressure sensitive paints may be used on test materials in wind tunnels as a way to map pressures without the use of pressure taps. Also, the degradation of oxygen in a wind tunnel flow (e.g., by increasing the amount of added nitrogen (versus decreasing)), would help the pressure sensitive paint luminescence.

In some embodiments, the test material may be exposed to the gaseous mixture (e.g., gaseous mixtures, nitrogenated gaseous mixtures, oxygenated gaseous mixtures) for a duration of contiguous and/or noncontiguous time. The duration of contiguous and/or noncontiguous time may vary depending on the specific test objectives and/or test material characteristics being evaluated. For example, the duration of contiguous time may be the duration or a portion of the duration of a simulated flight to determine how the test material may respond to conditions experienced during flight. In some embodiments, the test material may be exposed to different levels of nitrogen and/or oxygen for different time periods. For example, the levels of nitrogen and/or oxygen in the synthetic air may be modified to simulate flight conditions and the time period which the test material may experience these levels may be associated with the expected time that the test material will experience these levels during an actual flight. In these and other embodiments, the response of the test material to the gaseous mixture may be determined.

In some embodiments, determining the response of the test material may include determining oxidation behavior of the test material in response to exposing the test material to the gaseous mixture including those gaseous mixtures that have been nitrogenated and/or oxygenated with the addition of nitrogen gas and/or oxygen gas. In some embodiments, the determination of oxidation behavior may include measuring physical changes in the test material that may result from oxidation. For example, physical changes may include surface degradation, material loss, formation of oxide layers, changes in surface morphology, and/or changes in material thickness, among other physical changes. In some embodiments, the determination of oxidation behavior may include measuring chemical changes in the test material that may be attributable to oxidation. For example, chemical changes may include alterations in chemical composition, formation of new chemical compounds, and/or changes in molecular structure of the test material that may result from oxidation, among other chemical changes. In some embodiments, the determination of oxidation behavior may include measuring mechanical property changes in the test material that may result from oxidation. The mechanical property changes may include changes in tensile strength, changes in hardness, changes in elasticity, and//or changes in fracture toughness, among other mechanical property changes. In some embodiments, the determination of oxidation behavior may include measuring thermal property changes that may result from oxidation. The thermal property changes may include changes in thermal conductivity, changes in heat capacity, and/or changes in thermal expansion coefficient, among other thermal property changes.

In some embodiments, determination of oxidation behavior may include analyzing the degree of oxidation and/or rate of oxidation, among other metrics of oxidation. Determining the degree of oxidation may include determining the fraction of material oxidized (e.g., % mass change), the thickness of an oxide layer, and/or oxygen uptake per unit area, among other techniques of measuring an amount of oxidation. The determination of the rate of oxidation may include analyzing the degree of oxidation of the test material over the duration of exposure to the gaseous mixture or the determination of the degree of oxidation may include analyzing the rate of oxidation over the duration of exposure to the gaseous mixture. In these and other embodiments, the determination of oxidation behavior may involve the performance of analytical techniques such as thermogravimetric analysis (TGA), spectroscopy (e.g., X-ray photoelectron spectroscopy), microscopy (e.g., scanning electron microscopy), among other techniques.

In some embodiments, one or more operational adjustments may be made to the test material based on the determination of the oxidation behavior. For example, based on the degree of oxidation that may be determined, a thickness of the test material may be determined that may be configured to withstand the oxidation. For instance, the thickness of the test material may be determined such that the test material may be configured to withstand oxidation during an actual flight of a hypersonic vehicle that includes the test material.

In some embodiments, an oxidation model of the test material may be generated based on the oxidation behavior of the test material during exposure to the gaseous mixture including those that may have been nitrogenated or oxygenated. In these and other embodiments, the oxidation model may be developed through correlation of measured oxidation behavior with specific test conditions experienced by the test material during exposure to the gaseous mixture. In some embodiments, the oxidation model may incorporate relationships between oxygen concentration, exposure duration, temperature, pressure, and/or the resulting degree of oxidation observed in the test material. In some embodiments, the oxidation model may include empirical coefficients derived from the measured oxidation behavior data obtained during testing. These coefficients may be determined through regression analysis or curve fitting techniques, among other techniques, applied to the experimental data. In some embodiments, the oxidation model may account for the effects of transient oxygen atoms and transient nitrogen atoms present in the gaseous mixture, which may exhibit different reactivity compared to molecular oxygen and nitrogen.

In some embodiments, the oxidation model may be validated by comparing predicted oxidation behavior with additional experimental measurements performed under different test conditions. The validated model may then be used to extrapolate oxidation behavior to conditions that differ from those directly tested, such as longer exposure durations, different oxygen concentrations, and/or different nitrogen concentrations, among other conditions. In some embodiments, the oxidation model may be implemented in computational simulation tools that may predict material degradation over extended mission durations or under varying flight conditions. In these and other embodiments, the model may be refined iteratively as additional experimental data becomes available from subsequent testing of the test material under different conditions.

As an example use of the oxidation model, an oxygen gas concentration may be identified in the gaseous mixture and oxidation of the test material may be measured at the identified oxygen gas concentration and/or different oxygen gas concentrations that may be obtained by modifying the gaseous mixture to include the different oxygen gas concentrations (e.g., by modifying nitrogen gas flow to the test chamber and/or by modifying oxygen gas flow to the test chamber). An oxidation model may be generated based on the oxidation behavior at the different oxygen gas concentrations. In these and other embodiments, oxidation behavior of the test material may be estimated for potential deployment conditions using the oxidation model. Potential deployment conditions may include any conditions that may impact oxidation of the test material and which correspond to a potential use case of the test material (e.g., hypersonic flight conditions).

As another example use of the oxidation model, the degree of oxidation of the test material that may result from combustion heat flux may be determined. For example, the combustion heat flux may create localized high-temperature regions that may accelerate oxidation reactions, and the resulting oxidation products may be characterized using spectroscopic techniques to quantify the extent of oxidation. In these and other embodiments, the oxidation model may be generated and used to determine a predicted degree of oxidation resulting from combustion heat flux under predetermined conditions.

Thus, the gaseous mixture may be used as a proxy for air that may be experienced during actual flight conditions such that the test material may be physically evaluated under simulated flight conditions in a simulated flight environment such as a wind tunnel. The gaseous mixture may be modulated (e.g., flow characteristics, composition characteristics) for a time period (e.g., pre-determined time period or queued time period) to simulate conditions experienced during flight and oxidation of the test material may be monitored during the simulated flight to evaluate how the test material may respond to different air characteristics during flight. As a result, test materials may be adequately tested on the ground before actually being put into flight.

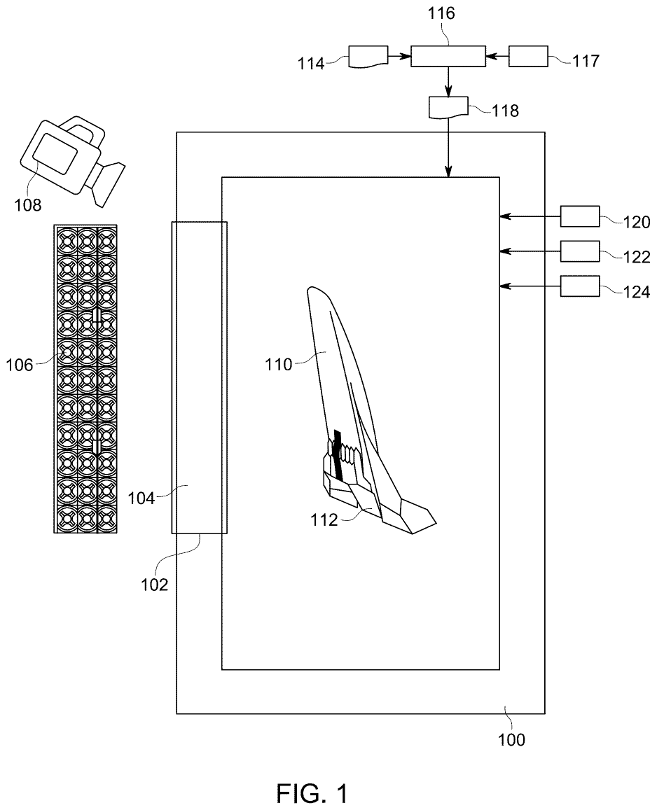

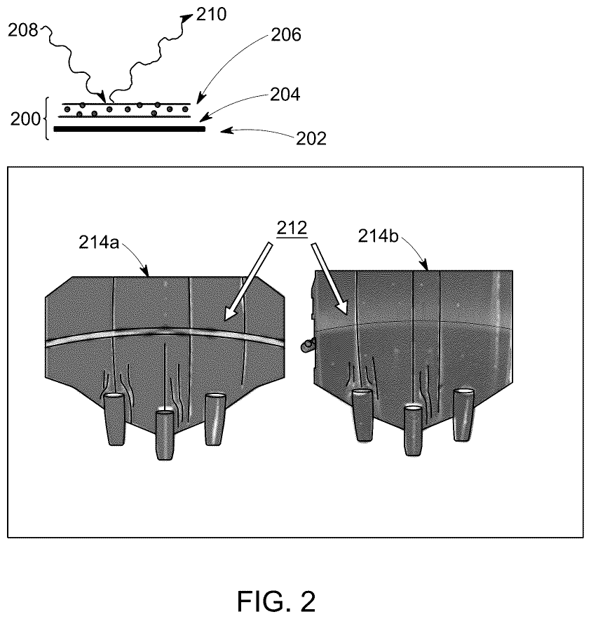

FIG. 1 illustrates a wind tunnel system that may be used in connection with controlling the nitrogen and/or oxygen content of synthetic air. The system includes a wind tunnel 100 having a window pane 102 having a window 104 therein that allows visual inspection of inside the wind tunnel 100, and which may allow visualization of the surface 110 of the object 112 being tested. The system includes an LED light 106 (e.g., UV LED array), which may be positioned or mounted so as to illuminate the surface 110 of the object 112 being tested. The system includes a camera 108, such as a video camera or still camera, which may capture images of the surface 110 of the object 112 being tested. For example, the LED light 106 may illuminate pressure sensitive paint on the surface 110 of the object 112 being tested as is described in more detail with reference to FIG. 2, and the camera 108 may image the illuminated pressure sensitive paint. For example, the LED light 106 may be configured to emit UV light, white light, IR light, or any specific wavelength or include a spectrum range.

In some embodiments, the wind tunnel and associated system is described in US Pat. App. Pub. No. 2022/0119256 A1 (U.S. application Ser. No. 17/502,527) entitled “NITROUS DECOMPOSITION WITHOUT A CATALYST,” which is incorporated herein in its entirety by specific reference.

In some embodiments, the ability to actively alter the incoming ratio of oxygen to nitrogen may be used to support research, development, verification and/or validation of propulsion engine analysis codes, material test coupons and methodologies. In some aspects, combustion delay, onset, stability, quench and/or stall margins may be analyzed empirically on the ground prior to any flight testing. This active alteration of the incoming ratio of oxygen to nitrogen may also be adjusted to expose the objects or systems to be tested to different experimental conditions (e.g., especially if there are dynamic actions, such as deployment of a surface such as a flap on a test article that coincides with a mission event and coordination of conditions is needed).

In some embodiments, the object 112 may be, include, and/or be composed of the test materials described throughout this disclosure. For example, the test material may be an engine included in the object 112. As another example, the object 112 may be a vehicle such as a rocket that may be considered as the test material. As an additional example, the object 112 may include test materials such as one or more refractory alloys, composites, and/or ceramics.

In an example operation of the system of FIG. 1, liquid nitrous 114 may be provided to a decomposition chamber 116 included in a decomposition system. The decomposition chamber 116 may be similar to the decomposition chamber described in US Pat. App. Pub. No. 2022/0119256 A1 (U.S. application Ser. No. 17/502,527), which is incorporated by reference herein. In the decomposition chamber 116, the liquid nitrous may be expanded and/or conditioned into gaseous nitrous, and the gaseous nitrous may obtain a temperature for decomposition as described in US Pat. App. Pub. No. 2022/0119256 A1 (U.S. application Ser. No. 17/502,527), which is incorporated by reference herein. In some embodiments, the temperature for decomposition may be at least about 900 degrees K.

In some embodiments, a heated nitrogen gas (N₂) 117 may be injected into the decomposition chamber 116 to heat the gaseous nitrous to a temperature for decomposition. After the temperature for decomposition has been obtained, the injection of the heated nitrogen gas (N2) 117 may be restricted in the decomposition chamber to increase a concentration of oxygen gas in the gaseous mixture 118. In some embodiments, the injection of the heated nitrogen gas 117 may be resumed after a period of time in which the injection of the heated nitrogen gas 117 was restricted.

A gaseous mixture 118 of oxygen gas (O₂), transient oxygen atoms (O), nitrogen gas (N₂), and/or transient nitrogen atoms (N) may be obtained from the decomposition of the nitrous in the decomposition chamber 116. The gaseous mixture 118 may be provided to a test chamber, which may be a portion of the wind tunnel 100. In some embodiments, the oxygen to nitrogen atom ratio may be about 1:2 in the gaseous mixture 118 (oxygen atoms/molar: nitrogen atoms/molar).

In some embodiments, the test chamber may be at least initially devoid of receiving an additional nitrogen gas (N₂) from a nitrogen gas supply 120 and/or from an air supply 122 and/or at least initially devoid of receiving additional oxygen gas (O₂) from the air supply 122 and/or from an oxygen supply 124.

In some embodiments, the additional nitrogen gas from the nitrogen gas supply 120 may be provided to the test chamber to nitrogenate the gaseous mixture 118. In some embodiments, the nitrogenated gaseous mixture may have a nitrogen content of less than or about 77% nitrogen gas (N₂) and transient nitrogen atoms (N) by volume or moles and greater than 22% oxygen gas (O₂) and transient oxygen atoms (O) by volume or moles. In some embodiments, the nitrogenated gaseous mixture may have greater than 80% nitrogen gas (N₂) and transient nitrogen atoms (N) by volume or moles and less than 21% oxygen gas (O₂) and transient oxygen atoms (O) by volume or moles. In some embodiments, the gaseous mixture 118 provided to the test chamber may include nitrogen gas (N₂) in the test chamber at between about 66% to about 77% by volume or moles or at between about 79% to about 95% volume or moles.

In some embodiments, the additional oxygen gas from the oxygen gas supply 124 may be provided to the test chamber to oxygenate the gaseous mixture 118. In some embodiments, the test chamber may be devoid of receiving additional oxygen gas from the oxygen gas supply 124 or optionally devoid of receiving additional oxygen gas from the oxygen gas supply 124.

In some embodiments, air from the air supply 122 may be provided to the test chamber. In some embodiments, the test chamber may be devoid or optionally devoid of receiving air from the air supply 122.

In some embodiments, the surface 112 of the object 110 being tested may be exposed to the gaseous mixture 118 with or without additional nitrogen gas from the nitrogen gas supply 120, air from the air supply 122, and/or additional oxygen gas from the oxygen gas supply 124 being provided to the wind tunnel 100. A response of the surface 112 (e.g., the test material) to the gaseous mixture 118 may be determined.

Thus, the wind tunnel 100 may allow for materials included in the object 110 to be tested according to various gaseous mixtures 118 in the wind tunnel 100, which may allow the object 110 to be tested on the ground with air conditions that simulate air conditions during flight. For example, oxidation behavior of the object 110 may be determined, which may allow for oxidation models to be generated for various test materials such that ground simulations may be similar to actual flight conditions. In some embodiments, pressure-sensitive paint may be applied to the object 110 and the response of the pressure-sensitive paint to the gaseous mixture 118 may be determined as is described in more detail with reference to FIG. 2.

FIG. 2 illustrates a pressure sensitive paint (PSP) measurement diagram. In some aspects, additional nitrogen may be added, which may yield an atmosphere of lower than 21% oxygen where PSP may be used. In some aspects, no additional nitrogen is added or additional oxygen may be added to yield an atmosphere of greater than 21% oxygen, as described herein. PSP is based on the oxygen-quenching phenomenon of luminescence of specific organic luminophores. For a given excitation level, the brightness of the luminescent material varies inversely with the partial pressure of oxygen and, hence, to the pressure of the air. When the PSP is applied to an aerodynamic surface and placed in an operating wind tunnel under appropriate lighting, the molecules luminesce as a function of the local pressure of oxygen over the surface of interest during aerodynamic flow. The resulting image will be brightest in the areas of low pressure (low oxygen concentration), and less intense in the areas of high pressure (where oxygen is most abundant on the surface). The luminescence data may be used to map a continuous pressure field over/under the model under investigation. As shown, the PSP allows for better visualization of the shock location and associated analysis.

In some embodiments, the use of the PSP may be performed in a wind tunnel where the system may vary the oxygen to nitrogen ratio. There is another application for the HOTTER approach, where nitrogen is intentionally introduced, and may yield an atmosphere of lower than 21% oxygen that will change the way pressure sensitive paint (PSP) may be used, or where no nitrogen is introduced or where oxygen is intentionally introduced to yield an atmosphere of greater than 21% oxygen.

The PSP analysis is based on the oxygen-quenching phenomenon of luminescence of specific organic luminophores. For a given excitation level, the brightness of the luminescent material varies inversely with the partial pressure of oxygen and, hence, to the pressure of the air. When the PSP is applied to an aerodynamic surface and placed in an operating wind tunnel under appropriate lighting, the molecules luminesce as a function of the local pressure of oxygen over the surface of interest during aerodynamic flow. The resulting image will be brightest in the areas of low pressure (low oxygen concentration), and less intense in the areas of high pressure (where oxygen is most abundant on the surface). The luminescence data may be used to map a continuous pressure field over/under the model under investigation. So there will be a difference in the way PSP may map flow fields for different nitrogen levels. Thus, PSP may be used in tunnels as a way to map pressures without the use of pressure taps. Also, any degradation of oxygen in a tunnel flow (e.g., by increasing the amount of added nitrogen (versus decreasing)) may facilitate PSP luminescence, thereby yielding better visualization.

As illustrated in FIG. 2, a test material 200 may have a test surface 202 to which a pressure sensitive paint having at least a first layer 204 and a second layer 206 may be applied. While the first layer 204 and the second layer 206 are described as separate layers for ease of description, it will be appreciated that these layers may form a single layer. The first layer 204 may be a base coat/primer layer. The first layer 204 may include a base coat of a paint (e.g., white paint) which may increase the reflection of light. In some embodiments, the first layer 204 may include or have an adhesive primer layer to facilitate adhesion of the first layer 204 to the test surface 202 and/or to facilitate adhesion of the first layer 204 and the second layer 206.

In these and other embodiments, the second layer 206 may include a polymer binder and oxygen-sensitive probe molecules. The oxygen-sensitive probe molecules may be organic luminophores, and the luminescence of the organic luminophores may be quenched by oxygen as described above.

As described previously, the pressure sensitive paint may cause the test material 200 to be optically responsive to light, and the emitted light may be used to determine areas that may correspond to low oxygen concentration, low oxygen pressure, high oxygen concentration, and/or high oxygen pressure.

In an example operation, an excitation wavelength 208 of light may be directed towards the test material 200. For example, the excitation wavelength 208 of light may be directed towards the test material 200 by an LED light such as the LED light 106 described with respect to FIG. 1. The excitation wavelength 208 may be ultraviolet light, visible light, infrared light, or another wavelength spectrum that may be configured to excite the oxygen-sensitive probe molecules in the second layer 206. The excitation wavelength 208 may be absorbed by the oxygen-sensitive probe molecules and the oxygen-sensitive probe molecules in the second layer 206 may output an emitted wavelength 210.

As described above, the emitted wavelength 210 may vary depending on the oxygen concentration at different areas of the test surface 202. In some embodiments, the emitted wavelength 210 may be characterized by variations in brightness across the test surface 202. Areas of the test surface 202 experiencing lower oxygen pressure or lower oxygen concentration may exhibit higher brightness in the emitted wavelength 210. Conversely, areas of the test surface 202 experiencing higher oxygen pressure or higher oxygen concentration may exhibit lower brightness in the emitted wavelength 210. The emitted wavelength 210 may be captured by an imaging device such as the camera 108 described with respect to FIG. 1. The imaging device may record the spatial distribution of brightness across the test surface 202.

The emitted wavelength 210 may be analyzed to determine and map a pressure field on the test surface 202. For example, FIG. 2 illustrates a first object 214a and a second object 214b, which may include the test material 200 having the test surface 202. The pressure-sensitive paint may be applied to the objects 214 at areas 212, which may correspond to regions of the test material 200. The objects 214 may then be exposed to various gaseous mixtures under various conditions as described throughout the present disclosure, and the pressure-sensitive paint may have different sub-areas of brightness depending on the oxygen-quenching that occurs in the different sub-areas. For example, the area 212 of the first object 214a illustrates white regions indicating a higher brightness and dark regions indicating a lower brightness. The white regions may be determined to have low oxygen concentration or low oxygen pressure as the brightness may be higher indicating that less oxygen quenching has occurred. The dark regions may be determined to have a high oxygen concentration or high oxygen pressure as the brightness may be lower thereby indicating that more oxygen quenching has occurred.

In these and other embodiments, a pressure field of the test surface 202 may be mapped based on the measured brightness. For example, the brightness distribution may be analyzed quantitatively by comparing measured luminescence intensities to calibration data that may relate brightness to oxygen partial pressure under known conditions. The calibration data may be obtained by exposing reference samples of the pressure sensitive paint to controlled oxygen concentrations and pressures in a calibration chamber prior to wind tunnel testing. A pressure field map may be generated by converting the spatial distribution of brightness measurements into corresponding pressure values using the calibration relationship. The pressure field map may reveal regions of flow separation, shock wave locations, and/or pressure gradients across the test surface 202, among other aerodynamic flow characteristics. Additionally or alternatively, the mapped pressure field may be used to identify locations on the test material 200 where elevated oxygen concentrations may lead to increased oxidation rates during actual flight conditions. As a result, the objects 212 may be modified based on the pressure field. For example, thickness of the test material 200 may be increased at areas that the pressure field may indicate have increased oxidation rates.

### Example

Air quality of the synthetic air as compared to atmospheric constituents has been evaluated (Table 1) using the following techniques. Sample was collected into gas cylinder, then assayed using Fourier-Transformed Infra-Red (FTIR) Spectroscopy and Gas Chromatograph (GC). The N₂O and NO concentration was measured in-situ using Tunable Laser Absorption Spectroscopy (TLAS) to identify quantity of N₂O and NO. All relevant contaminants were molecules comprising Nitrogen and Oxygen. There were no Hydrocarbon Combustion Products (CO₂, H₂O) representing a feature of the creation of synthetic air as is described in U.S. Pat. App. Pub. 2022/0119256 A1 (U.S. application Ser. No. 17/502,527) entitled “NITROUS DECOMPOSITION WITHOUT A CATALYST.”

The following Table 1 shows that the process described herein may be used to deliver clean air including nitrogen and oxygen species molecules generated and/or encountered in hypersonic flight.

TABLE 1

| O₂ | N₂ | N₂O | NO | NO₂ | Measurement Method |
|---|---|---|---|---|---|
| 24% | 72% | 0.58% | 0.01% | 2.23% | Sample Cylinder, FTIR/GC |
| 20% | 76% | 0.40% | 0.01% | 3.03% | Sample Cylinder, FTIR/GC |
| | | 0.06% | 1.77% | | In-Situ TLAS |
| | | 0.13% | 1.94% | | In-Situ TLAS |

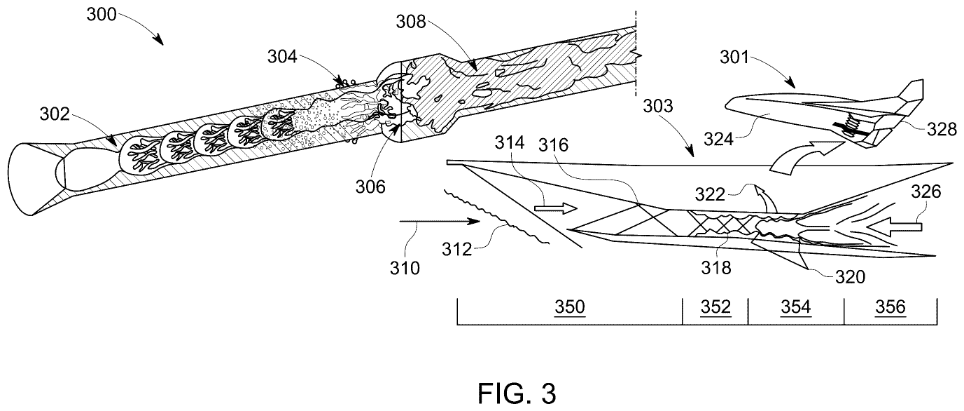

FIG. 3 shows an example of a testing paradigm where a Thermal Protection System (TPS) construct called acreage exists, which protects the inner flow paths of the scramjet engine 303 of the hypersonic vehicle 301. A testing apparatus 300 is depicted above the flight vehicle schematic. This testing needs to be done on the ground to determine the TPS survivability, operability, sustainability, and/or maintainability prior to installation into an expensive flight test. For example, the design margins may be verified prior to flight using the pressure sensitive paint system. The data values determined from ground testing may be used, along with their uncertainties, such that modeling and simulations may be performed on a computing system. The modeling and simulations may be verified and validated with the test data using the PSP system.

The example testing apparatus 300 illustrates a shock train 302, fuel injection 304, a flame front 306, and product gases 308. Referring to FIG. 3, the shock train 302 may be configured to simulate a series of shock waves that may form in the supersonic flow path of the scramjet engine 303. The shock train 302 may decelerate the incoming high-speed flow to facilitate combustion stability. The fuel injection 304 may introduce fuel into the flow stream at a location downstream of the shock train 302. The fuel injection 304 may be accomplished through one or more injectors that may be positioned along the combustor wall or within the flow path. The flame front 306 may represent the region where combustion reactions may occur between the injected fuel and the oxygen present in the gaseous mixture. The product gases 308 may be the combustion products that may result from the chemical reactions at the flame front 306. The product gases 308 may include water vapor, carbon dioxide, and other species depending on the fuel composition. The product gases 308 may expand and accelerate through the nozzle section of the scramjet engine 303 to generate thrust.

As illustrated in FIG. 3, the engine 303 of the hypersonic vehicle 301 may be an air-breathing engine such as a scramjet engine. The engine 303 may include an inlet 350, an isolator 352, a combustor 354, and a nozzle 356. In operation, air flow 310 may be ingested by the engine 303 via the inlet 350. The air flow 310 may include air having various mixtures of nitrogen gas, oxygen gas, atomic oxygen, and/or atomic nitrogen depending on the flight regime. The effect of these gaseous mixtures on the engine 303 and/or the vehicle 301 may be simulated on the ground via the systems and/or methods described throughout this disclosure. The airflow 310 can be generated as described herein.

The air flow 310 entering the engine 303 may create a bow shock 312 as the air interacts with the forebody 324 of the vehicle 301. The forebody 324 may be shaped and configured to cause the air flow 310 to be compressed as the air flow enters the inlet 350. Moreover, the airflow 310 entering the engine 303 via the inlet 350 causes drag 314. As the air flow 312 enters the isolator 352 shock boundary layer interactions 316 may occur. The air flow 310 may enter the isolator 352 where an isolator shock train 318 may be induced. The air flow 310 may exit the isolator 352 and enter the combustor 354 where fuel injection 320 may occur and the mixture of air and fuel may be combusted. As the mixture of air and fuel is combusted, heat loss 322 may occur. The product gases 308 generated from the combustion may be expelled from the engine 303 via the nozzle 356 which may generate thrust 326. The aftbody 328 of the vehicle may be shaped and configured to allow the gases created in the combustor 354 to expand.

The testing apparatus 300 and the hypersonic vehicle 301 are illustrated and described to demonstrate example objects that may be ground tested via the systems and methods described herein to simulate air experienced during hypersonic flight. For example, materials used in the hypersonic vehicle 301 may be ground tested to determine the response of these materials before testing these materials in flight.

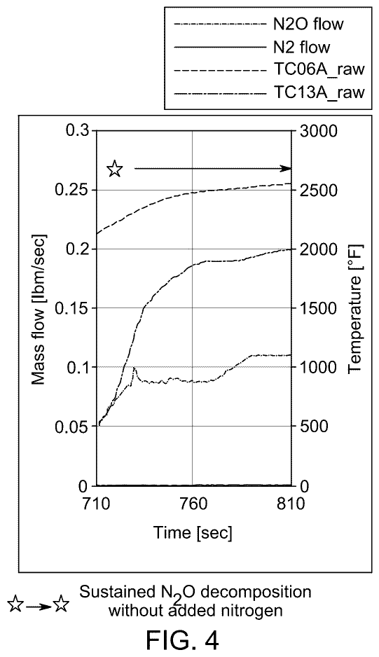

FIG. 4 shows data from a test in the hypersonic research tunnel known as the Hypersonic Synthetic Environment Test Tunnel (HySETT) where a higher concentration of oxygen (over what is found in ambient air) was produced, showing the technical feasibility of the concept of creating a Hypersonic Oxidization Thermal Testing with Empirical Research (HOTTER) ground test environment, in which oxidation of test materials may be determined on the ground. This may allow models simulating hypersonic flight to be modified based on the ground testing results from the methods and/or systems described herein. As a result, the models may be improved, which may allow for improved understanding of test material responses resulting from hypersonic flight such as aging due to oxidation. The data is from an example run with pure N₂O flow in the HySETT, which results in decomposition into outlet test gas of two (2) molar parts N₂ and one (1) molar part O₂ at high temperatures. In the example illustrated in FIG. 4, no molecular nitrogen was added for about 100 seconds. The graph shows the flows and temperatures for the run prior to and after the N₂ is added to make synthetic air (e.g., synthetic gas representing air).

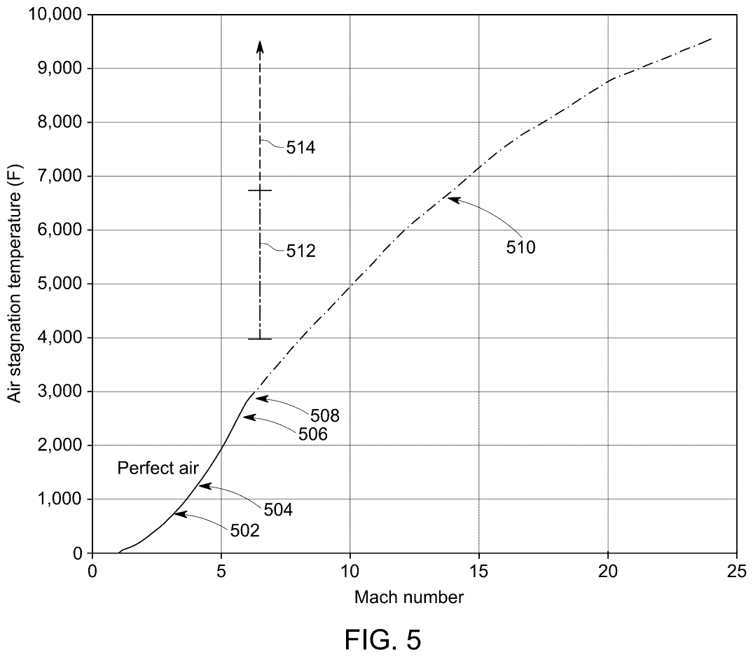

Generally, FIG. 5 includes a classic temperature chart (Bertin, John J. Hypersonic Aerothermodynamics. Washington, DC: American Institute of Aeronautics and Astronautics, 1994) of atmospheric air qualities experienced in different flight regimes. FIG. 5 shows the conditions at altitude a vehicle travelling at hypersonic conditions would experience, with some of the regions of atomic dissociation noted. A “perfect air” region 502 is illustrated as a solid line in which illustrates the limit of temperature corresponding to enthalpy release due to decomposition of nitrous oxide. As illustrated in FIG. 5, the perfect air region 502 ends at about 2985 degrees Fahrenheit at which point the nitrous oxide in the air begins to decompose. In the perfect air region 502, several melting points are also indicated for reference. For example, the melting point of aluminum 504, the melting point of Steel/Inconel 506, and the melting point of titanium 508 are also shown.

A high temperature air region 510 is also illustrated which corresponds to the dashed portion of the line graph following the perfect air region 502. As illustrated in FIG. 5, the high temperature air region 510 may include regions of oxygen dissociation 512 and nitrogen dissociation 514. These regions illustrate temperatures at which oxygen and nitrogen may begin to dissociation.

As illustrated, in FIG. 5, above about Mach 5, the thermal environment for hypersonic flight drives the need for vehicle thermal protection systems. The nitrous oxide decomposition alone may yield about Mach 6, to get higher super heating nitrogen gas is added. As shown, the data shows the capability to modulate or sustain an oxygen rich synthetic air environment for testing exposure of materials and systems to harsh conditions.

### EMBODIMENTS

### Embodiment 1

A method of controlling nitrogen and oxygen content of synthetic air, comprising:
decomposing nitrous in a decomposition system by expanding liquid nitrous into gaseous nitrous in a decomposition chamber of the decomposition system, wherein the gaseous nitrous obtains a temperature for decomposition;
obtaining a gaseous mixture of oxygen gas (O₂), transient oxygen atoms (O), nitrogen gas (N₂), and/or transient nitrogen atoms (N) from the decomposition of the nitrous, wherein the oxygen atom to nitrogen atom ratio is about 1:2 (oxygen atoms/molar: nitrogen atoms/molar);
providing the gaseous mixture to a test chamber, wherein the test chamber is at least initially devoid of receiving an additional nitrogen gas (N₂) from either an N₂ supply or a reservoir of air including both N₂ and O₂ and/or at least initially devoid of receiving additional oxygen gas (O₂) from the reservoir of air including both N₂ and O₂;
exposing a test material to the gaseous mixture in the test chamber; and
determining a response of the test material to the gaseous mixture.

### Embodiment 2

The method of Embodiment 1, wherein the test chamber is initially devoid of receiving the additional nitrogen gas.

### Embodiment 3

The method of Embodiments 1 or 2, wherein the test chamber is devoid of receiving the additional nitrogen gas (N₂) or transient nitrogen atoms (N) and the method further comprises providing the additional oxygen gas (O₂) and/or transient oxygen atoms (O) into the test chamber.

### Embodiment 4

The method of Embodiment 1, wherein the method further comprises:
providing the additional nitrogen gas (N2) into the test chamber to obtain a nitrogenated gaseous mixture including the additional nitrogen gas (N2) and the gaseous mixture, wherein the test chamber is devoid of receiving the additional oxygen gas (O₂), wherein the nitrogenated gaseous mixture has a nitrogen content of either:
   less than or about 77% nitrogen gas (N2) and transient nitrogen atoms (N) by volume or moles and greater than 22% oxygen gas (O₂) and transient oxygen atoms (O) by volume or moles; or
   greater than 80% nitrogen gas nitrogen gas (N2) and transient nitrogen atoms (N) by volume or moles and less than 21% oxygen gas (O2) and transient oxygen atoms (O) by volume or moles;
wherein exposing the test material in the test chamber to the gaseous mixture includes exposing the test material to the nitrogenated gaseous mixture; and
wherein determining the response of the test material includes determining the response of the test material to the nitrogenated gaseous mixture.

### Embodiment 5

The method of any of the Embodiments, wherein the gaseous mixture or a nitrogenated gaseous mixture generated from the gaseous mixture and additional nitrogen gas (N2) provided to the test chamber includes nitrogen gas (N2) in the test chamber at between about 66% to about 77% by volume or moles or at between about 79% to about 95% by volume or moles.

### Embodiment 6

The method of any of the Embodiments, further comprising:
exposing the test material to the gaseous mixture or a nitrogenated gaseous mixture generated from the gaseous mixture and the additional nitrogen gas (N2) for a duration of contiguous and/or noncontiguous time, wherein determining the response of the test material to the gaseous mixture includes determining oxidation behavior of the test material in response to exposing the test material to the gaseous mixture or the nitrogenated gaseous mixture; and generating an oxidation model of the test material based on the oxidation behavior of the test material over the duration of contiguous and/or noncontiguous time.

### Embodiment 7

The method of any of the Embodiments, wherein the test material includes at least one of part of a thermal protection system of a plane, missile, rocket, projectile, carrier, flying object, or drone, an engine, wherein the method optionally includes performing an engine combustion test, rocket fuel material, a SiC composite, a ZrB2 composite, a ZrB2-SiC composite, a ZrB2-ZrC—SiC composite, a refractory alloy, a composite, or a ceramic.

### Embodiment 8

The method of any of the Embodiments, wherein determining the response of the test material to the gaseous mixture includes determining a degree of oxidation of the test material resulting from being exposed to the gaseous mixture.

### Embodiment 9

The method of any of the Embodiments, wherein the method further comprises determining a thickness of the test material configured to withstand the degree of oxidation.

### Embodiment 10

The method of any of the Embodiments, further comprising:
simulating flight conditions in a simulated flight environment using the gaseous mixture as a proxy for air in the flight conditions;
modulating flow characteristics of the gaseous mixture for a determined time period or queued time periods; and
monitoring oxidation of the test material during the determined time period or queued time periods.

### Embodiment 11

The method of any of the Embodiments, further comprising:
identifying an oxygen gas (O2) concentration in the gaseous mixture;
measuring oxidation of the test material at different oxygen gas (O2) concentrations by modifying the gaseous mixture to include the different oxygen gas (O2) concentrations;
generating an oxidation model of the test material based on the oxidation behavior at the different oxygen gas (O2) concentrations; and
estimating oxidation behavior of the test material for potential deployment conditions using the oxidation model.

### Embodiment 12

The method of any of the Embodiments, wherein decomposing the nitrous includes:
conditioning liquid nitrous into gaseous nitrous in the decomposition chamber;
injecting heated nitrogen gas (N2) into the decomposition chamber so as to mix with and/or modulate the gaseous nitrous, wherein the injection of the heated nitrogen gas (N2) is regulated to obtain the temperature for decomposition of the gaseous nitrous,
heating the gaseous nitrous to the temperature for decomposition of the gaseous nitrous; decomposing the gaseous nitrous into the gaseous mixture, the gaseous mixture including the nitrogen gas (N2) and the oxygen gas (O2);
restricting injection of the heated nitrogen gas into the decomposition chamber to increase a concentration of the oxygen gas (O2) in the gaseous mixture; and
analyzing the test material in the test chamber based on the increased concentration of oxygen gas (O2) in the gaseous mixture.

### Embodiment 13

The method of any of the Embodiments, comprising:
injecting heated nitrogen gas (N2) into the decomposition chamber for a first period of time; restricting or terminating injection of the heated nitrogen gas (N2) into the decomposition chamber for a second period of time after the first period of time;
optionally, reinjecting the heated nitrogen gas (N2) into the decomposition chamber for a third period of time after the second period of time; and
determining oxidation of the test material in the test chamber during the second period of time and/or third period of time.

### Embodiment 14

The method of any of the Embodiments, comprising heating the nitrous to at least the temperature for decomposition, the temperature for decomposition being at least about 900 degrees K.

### Embodiment 15

The method of any of the Embodiments, wherein determining the response of the test material includes determining a degree of oxidation of the test material resulting from combustion heat flux and wherein the method further comprises:
generating an oxidation model of the test material based on the response of the test material; and
determining a predicted degree of oxidation resulting from combustion heat flux under predetermined conditions based on the oxidation model.

### Embodiment 16

The method of any of the Embodiments, further comprising:
monitoring oxidation of the test material in the test chamber; and
modeling the oxidation of the test material for different concentrations of oxygen and nitrogen in the gaseous mixture.

### Embodiment 17

The method of any of the Embodiments, wherein a pressure sensitive paint is applied to a test surface of the test material before exposing the test material to the gaseous mixture and the method further comprises:
measuring brightness of luminescence of the pressure sensitive paint in response to the test material being exposed to the gaseous mixture;
determining areas of high brightness to have low oxygen concentration or low oxygen pressure; determining areas of low brightness to have high oxygen concentration or high oxygen pressure; and
mapping a continuous pressure field of the test surface based on the measured brightness.

### Embodiment 18

The method of any of the Embodiments, wherein the gaseous mixture has a nitrogen concentration higher than ambient air and an oxygen concentration of less than 21% by volume or moles when the test material is exposed to the gaseous mixture.

### Embodiment 19

A method of controlling nitrogen and oxygen content of synthetic air, comprising:
decomposing nitrous in a decomposition system by expanding liquid nitrous into gaseous nitrous in a decomposition chamber of the decomposition system, wherein the gaseous nitrous obtains a temperature for decomposition;
obtaining a gaseous mixture of oxygen gas (O₂), transient oxygen atoms (O), nitrogen gas (N2), and/or transient nitrogen atoms (N) from the decomposition of the nitrous, wherein the oxygen atom to nitrogen atom ratio is about 1:2 (oxygen atoms/molar: nitrogen atoms/molar);
providing the gaseous mixture to a test chamber;
providing an additional nitrogen gas (N2) into the test chamber to obtain a nitrogenated gaseous mixture, wherein the test chamber optionally receives an additional oxygen gas (O2), wherein the nitrogenated gaseous mixture has a nitrogen content of either:
   less than or about 77% nitrogen gas (N2) and transient nitrogen atoms (N) by volume or moles and greater than 22% oxygen gas (O2) and transient oxygen atoms (O) by volume or moles; or
   greater than 80% nitrogen gas nitrogen gas (N2) and transient nitrogen atoms (N) by volume or moles and less than 21% oxygen gas (O2) and transient oxygen atoms (O) by volume or moles;
exposing a test material to the nitrogenated gaseous mixture in the test chamber; and
determining a response of the test material to the nitrogenated gaseous mixture.

### Embodiment 20

The method of Embodiment 19, wherein a pressure sensitive paint is applied to a test surface of the test material before exposing the test material to the gaseous mixture and the method further comprises:
measuring brightness of luminescence of the pressure sensitive paint in response to the test material being exposed to the gaseous mixture;
determining areas of high brightness to have low oxygen concentration or low oxygen pressure; determining areas of low brightness to have high oxygen concentration or high oxygen pressure; and
mapping a continuous pressure field of the test surface based on the measured brightness.

### Embodiment 21

A method of controlling nitrogen and oxygen content of synthetic air, comprising:
decomposing nitrous in a decomposition system by conditioning liquid nitrous into gaseous nitrous in a decomposition chamber of the decomposition system, wherein the gaseous nitrous obtains a temperature for decomposition;
obtaining a gaseous mixture of oxygen gas (O2), transient oxygen atoms (O), nitrogen gas (N2), and/or transient nitrogen atoms (N) from the decomposition, wherein the oxygen atom to nitrogen atom ratio is about 1:2 (oxygen atoms/molar: nitrogen atoms/molar);
providing the gaseous mixture to a test chamber;
providing an additional nitrogen gas (N2) into the test chamber to obtain a nitrogenated gaseous mixture, wherein the test chamber is devoid of receiving additional oxygen gas (O2) input, wherein the nitrogenated gaseous mixture has a nitrogen content of either:
   less than or about 77% nitrogen gas (N2) and transient nitrogen atoms (N) by volume or moles and greater than 22% oxygen gas (O2) and transient oxygen atoms (O) by volume or moles; or
   greater than 80% nitrogen gas nitrogen gas (N2) and transient nitrogen atoms (N) by volume or moles and less than 21% oxygen gas (O2) and transient oxygen atoms (O) by volume or moles;
exposing the nitrogenated gaseous mixture to a test material in the test chamber; and
determining a response of the test material to the nitrogenated gaseous mixture.

### Embodiment 22

The method of Embodiment 21, wherein a pressure sensitive paint is applied to a test surface of the test material before exposing the test material to the gaseous mixture and the method further comprises:
measuring brightness of luminescence of the pressure sensitive paint in response to the test material being exposed to the gaseous mixture;
determining areas of high brightness to have low oxygen concentration or low oxygen pressure; determining areas of low brightness to have high oxygen concentration or high oxygen pressure; and
mapping a continuous pressure field of the test surface based on the measured brightness.

One skilled in the art will appreciate that, for the processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

In one embodiment, the present methods may include aspects performed on a computing system. As such, the computing system may include a memory device that has the computer-executable instructions for performing the methods. The computer-executable instructions may be part of a computer program product that includes one or more protocols or algorithms for performing any of the methods of any of the claims.

In one embodiment, any of the operations, processes, or methods, described herein may be performed or cause to be performed in response to execution of computer-readable instructions stored on a computer-readable medium and executable by one or more processors. The computer-readable instructions may be executed by a processor of a wide range of computing systems from desktop computing systems, portable computing systems, tablet computing systems, hand-held computing systems, as well as network elements, and/or any other computing device.

The computer readable medium is not transitory. The computer readable medium is a physical medium having the computer-readable instructions stored therein so as to be physically readable from the physical medium by the computer/processor.

There are various vehicles by which processes and/or systems and/or other technologies described herein may be effected (e.g., hardware, software, and/or firmware), and that the preferred vehicle may vary with the context in which the processes and/or systems and/or other technologies are deployed. For example, if an implementer determines that speed and accuracy are paramount, the implementer may opt for a mainly hardware and/or firmware vehicle; if flexibility is paramount, the implementer may opt for a mainly software implementation; or, yet again alternatively, the implementer may opt for some combination of hardware, software, and/or firmware.

The various operations described herein may be implemented, individually and/or collectively, by a wide range of hardware, software, firmware, or virtually any combination thereof. In one embodiment, several portions of the subject matter described herein may be implemented via application specific integrated circuits (ASICs), field programmable gate arrays (FPGAs), digital signal processors (DSPs), or other integrated formats. However, some aspects of the embodiments disclosed herein, in whole or in part, may be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and/or firmware are possible in light of this disclosure. In addition, the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a physical signal bearing medium include, but are not limited to, the following: a recordable type medium such as a floppy disk, a hard disk drive (HDD), a compact disc (CD), a digital versatile disc (DVD), a digital tape, a computer memory, or any other physical medium that is not transitory or a transmission. Examples of physical media having computer-readable instructions omit transitory or transmission type media such as a digital and/or an analog communication medium (e.g., a fiber optic cable, a waveguide, a wired communication link, a wireless communication link, etc.).

It is common to describe devices and/or processes in the fashion set forth herein, and thereafter use engineering practices to integrate such described devices and/or processes into data processing systems. That is, at least a portion of the devices and/or processes described herein may be integrated into a data processing system via a reasonable amount of experimentation. A typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems, including feedback loops and control motors (e.g., feedback for sensing position and/or velocity; control motors for moving and/or adjusting components and/or quantities). A typical data processing system may be implemented utilizing any suitable commercially available components, such as those generally found in data computing/communication and/or network computing/communication systems.

The herein described subject matter sometimes illustrates different components contained within, or connected with, different other components. Such depicted architectures are merely exemplary, and that in fact, many other architectures may be implemented which achieve the same functionality. In a conceptual sense, any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermedial components. Likewise, any two components so associated may also be viewed as being “operably connected”, or “operably coupled”, to each other to achieve the desired functionality, and any two components capable of being so associated may also be viewed as being “operably couplable”, to each other to achieve the desired functionality. Specific examples of operably couplable include, but are not limited to: physically mateable and/or physically interacting components and/or wirelessly interactable and/or wirelessly interacting components and/or logically interacting and/or logically interactable components.

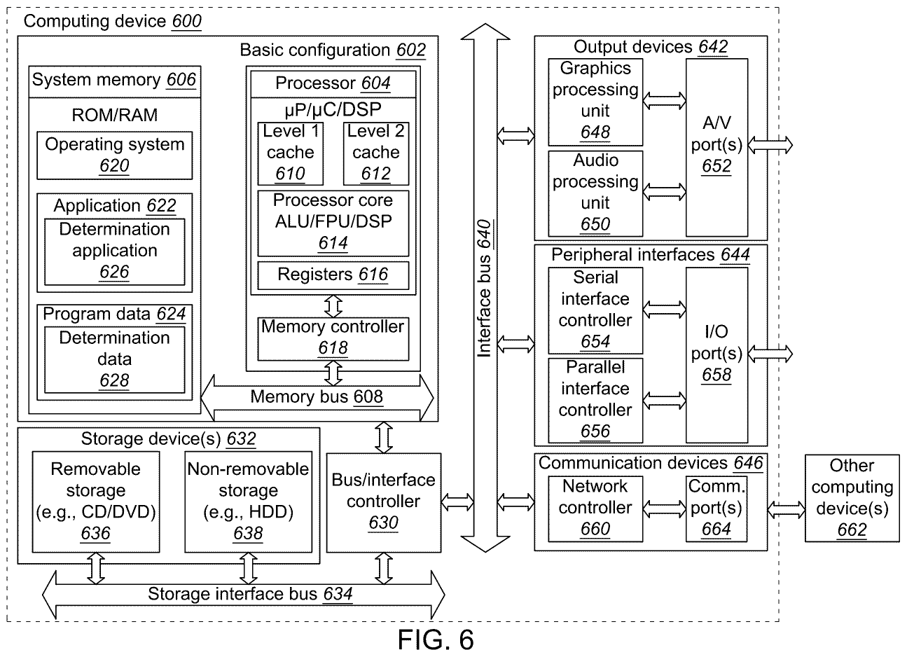

FIG. 6 shows an example computing device 600 (e.g., a computer) that may be arranged in some embodiments to perform the methods (or portions thereof) described herein. In a very basic configuration 602, computing device 600 generally includes one or more processors 604 and a system memory 606. A memory bus 608 may be used for communicating between processor 604 and system memory 606.

Depending on the desired configuration, processor 604 may be of any type including, but not limited to: a microprocessor (μP), a microcontroller (μC), a digital signal processor (DSP), or any combination thereof. Processor 604 may include one or more levels of caching, such as a level one cache 610 and a level two cache 612, a processor core 614, and registers 616. An example processor core 614 may include an arithmetic logic unit (ALU), a floating point unit (FPU), a digital signal processing core (DSP Core), or any combination thereof. An example memory controller 618 may also be used with processor 604, or in some implementations, memory controller 618 may be an internal part of processor 604.

Depending on the desired configuration, system memory 606 may be of any type including, but not limited to: volatile memory (such as RAM), non-volatile memory (such as ROM, flash memory, etc.), or any combination thereof. System memory 606 may include an operating system 620, one or more applications 622, and program data 624. Application 622 may include a determination application 626 that is arranged to perform the operations as described herein, including those described with respect to methods described herein. The determination application 626 may obtain data, such as pressure, flow rate, and/or temperature, and then determine a change to the system to change the pressure, flow rate, and/or temperature.

Computing device 600 may have additional features or functionality, and additional interfaces to facilitate communications between basic configuration 602 and any required devices and interfaces. For example, a bus/interface controller 630 may be used to facilitate communications between basic configuration 602 and one or more data storage devices 632 via a storage interface bus 634. Data storage devices 632 may be removable storage devices 636, non-removable storage devices 638, or a combination thereof. Examples of removable storage and non-removable storage devices include: magnetic disk devices such as flexible disk drives and hard-disk drives (HDD), optical disk drives such as compact disk (CD) drives or digital versatile disk (DVD) drives, solid state drives (SSD), and tape drives to name a few. Example computer storage media may include: volatile and non-volatile, removable and non-removable media implemented in any method or technology for storage of information, such as computer readable instructions, data structures, program modules, or other data.

System memory 606, removable storage devices 636 and non-removable storage devices 638 are examples of computer storage media. Computer storage media includes, but is not limited to: RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by computing device 600. Any such computer storage media may be part of computing device 600.

Computing device 600 may also include an interface bus 640 for facilitating communication from various interface devices (e.g., output devices 642, peripheral interfaces 644, and communication devices 646) to basic configuration 602 via bus/interface controller 630. Example output devices 642 include a graphics processing unit 648 and an audio processing unit 650, which may be configured to communicate to various external devices such as a display or speakers via one or more A/V ports 652. Example peripheral interfaces 644 include a serial interface controller 654 or a parallel interface controller 656, which may be configured to communicate with external devices such as input devices (e.g., keyboard, mouse, pen, voice input device, touch input device, etc.) or other peripheral devices (e.g., printer, scanner, etc.) via one or more I/O ports 658. An example communication device 646 includes a network controller 660, which may be arranged to facilitate communications with one or more other computing devices 662 over a network communication link via one or more communication ports 664.

The network communication link may be one example of a communication media. Communication media may generally be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR), and other wireless media. The term computer readable media as used herein may include both storage media and communication media.

Computing device 600 may be implemented as a portion of a small-form factor portable (or mobile) electronic device such as a cell phone, a personal data assistant (PDA), a personal media player device, a wireless web-watch device, a personal headset device, an application specific device, or a hybrid device that includes any of the above functions. Computing device 600 may also be implemented as a personal computer including both laptop computer and non-laptop computer configurations. The computing device 600 may also be any type of network computing device. The computing device 600 may also be an automated system as described herein.

The embodiments described herein may include the use of a special purpose or general-purpose computer including various computer hardware or software modules.

Embodiments within the scope of the present invention also include computer-readable media for carrying or having computer-executable instructions or data structures stored thereon. Such computer-readable media may be any available media that may be accessed by a general purpose or special purpose computer. By way of example, and not limitation, such computer-readable media may comprise RAM, ROM, EEPROM, CD-ROM or other optical disk storage, magnetic disk storage or other magnetic storage devices, or any other medium which may be used to carry or store desired program code means in the form of computer-executable instructions or data structures and which may be accessed by a general purpose or special purpose computer. When information is transferred or provided over a network or another communications connection (either hardwired, wireless, or a combination of hardwired or wireless) to a computer, the computer properly views the connection as a computer-readable medium. Thus, any such connection is properly termed a computer-readable medium. Combinations of the above should also be included within the scope of computer-readable media.

Computer-executable instructions comprise, for example, instructions and data which cause a general purpose computer, special purpose computer, or special purpose processing device to perform a certain function or group of functions. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

In some embodiments, a computer program product may include a non-transient, tangible memory device having computer-executable instructions that when executed by a processor, cause performance of a method that may include: providing a dataset having object data for an object and condition data for a condition; processing the object data of the dataset to obtain latent object data and latent object-condition data with an object encoder; processing the condition data of the dataset to obtain latent condition data and latent condition-object data with a condition encoder; processing the latent object data and the latent object-condition data to obtain generated object data with an object decoder; processing the latent condition data and latent condition-object data to obtain generated condition data with a condition decoder; comparing the latent object-condition data to the latent-condition data to determine a difference; processing the latent object data and latent condition data and one of the latent object-condition data or latent condition-object data with a discriminator to obtain a discriminator value; selecting a selected object from the generated object data based on the generated object data, generated condition data, and the difference between the latent object-condition data and latent condition-object data; and providing the selected object in a report with a recommendation for validation of a physical form of the object. The non-transient, tangible memory device may also have other executable instructions for any of the methods or method steps described herein. Also, the instructions may be instructions to perform a non-computing task, such as synthesis of a molecule and or an experimental protocol for validating the molecule. Other executable instructions may also be provided.

In some embodiments, a computer program product may include a non-transient, tangible memory device having computer-executable instructions that when executed by a processor, cause performance of a method described herein.

The non-transient, tangible memory device may also have other executable instructions for any of the methods or method steps described herein. Also, the instructions may be instructions to perform a non-computing task, such as synthesis of a molecule and or an experimental protocol for validating the molecule. Other executable instructions may also be provided.

The present disclosure is not to be limited in terms of the particular embodiments described in this application, which are intended as illustrations of various aspects. Many modifications and variations may be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art may translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range may be easily recognized as sufficiently describing and enabling the same range being broken down into at least equal halves, thirds, quarters, fifths, tenths, etc. As a non-limiting example, each range discussed herein may be readily broken down into a lower third, middle third and upper third, etc. As will also be understood by one skilled in the art all language such as “up to,” “at least,” and the like include the number recited and refer to ranges which may be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

All references recited herein are incorporated herein by specific reference in their entirety: U.S. Pat. No. 11,779,882; WO 2020/210179; US 2022/0119256; and U.S. Pat. No. 9,958,323.

1. A method of controlling nitrogen and oxygen content of synthetic air, comprising:
decomposing nitrous in a decomposition system by expanding liquid nitrous into gaseous nitrous in a decomposition chamber of the decomposition system, wherein the gaseous nitrous obtains a temperature for decomposition;
obtaining a gaseous mixture of oxygen gas (O₂), transient oxygen atoms (O), nitrogen gas (N₂), and/or transient nitrogen atoms (N) from the decomposition of the nitrous, wherein the oxygen atom to nitrogen atom ratio is about 1:2 (oxygen atoms/molar: nitrogen atoms/molar);
providing the gaseous mixture to a test chamber, wherein the test chamber is at least initially devoid of receiving an additional nitrogen gas (N₂) from either an N₂ supply or a reservoir of air including both N₂ and O₂ and/or at least initially devoid of receiving additional oxygen gas (O₂) from the reservoir of air including both N₂ and O₂;
exposing a test material to the gaseous mixture in the test chamber; and
determining a response of the test material to the gaseous mixture.

2. The method of claim 1, wherein the test chamber is initially devoid of receiving the additional nitrogen gas (N₂).

3. The method of claim 1, wherein the test chamber is devoid of receiving the additional nitrogen gas (N₂) or transient nitrogen atoms (N) and the method further comprises providing the additional oxygen gas (O₂) and/or transient oxygen atoms (O) into the test chamber.

4. The method of claim 1, wherein the method further comprises:
providing the additional nitrogen gas (N₂) into the test chamber to obtain a nitrogenated gaseous mixture including the additional nitrogen gas (N₂) and the gaseous mixture, wherein the test chamber is devoid of receiving the additional oxygen gas (O₂), wherein the nitrogenated gaseous mixture has a nitrogen content of either:
less than or about 77% nitrogen gas (N₂) and transient nitrogen atoms (N) by volume or moles and greater than 22% oxygen gas (O₂) and transient oxygen atoms (O) by volume or moles; or
greater than 80% nitrogen gas nitrogen gas (N₂) and transient nitrogen atoms (N) by volume or moles and less than 21% oxygen gas (O₂) and transient oxygen atoms (O) by volume or moles;
wherein exposing the test material in the test chamber to the gaseous mixture includes exposing the test material to the nitrogenated gaseous mixture; and
wherein determining the response of the test material includes determining the response of the test material to the nitrogenated gaseous mixture.

5. The method of claim 1, wherein the gaseous mixture or a nitrogenated gaseous mixture generated from the gaseous mixture and additional nitrogen gas (N₂) provided to the test chamber includes nitrogen gas (N₂) in the test chamber at between about 66% to about 77% by volume or moles or at between about 79% to about 95% by volume or moles.

6. The method of claim 1, further comprising:
exposing the test material to the gaseous mixture or a nitrogenated gaseous mixture generated from the gaseous mixture and the additional nitrogen gas (N₂) for a duration of contiguous and/or noncontiguous time, wherein determining the response of the test material to the gaseous mixture includes determining oxidation behavior of the test material in response to exposing the test material to the gaseous mixture or the nitrogenated gaseous mixture; and
generating an oxidation model of the test material based on the oxidation behavior of the test material over the duration of contiguous and/or noncontiguous time.

7. The method of claim 1, wherein the test material includes at least one of:
part of a thermal protection system of a plane, missile, rocket, projectile, carrier, flying object, or drone;
an engine, wherein the method optionally includes performing an engine combustion test;
rocket fuel material;
a SiC composite;
a ZrB₂ composite;
a Zr B₂—SiC composite;
a ZrB₂—ZrC—SiC composite;
a refractory alloy;
a composite; or
a ceramic.

8. The method of claim 1, wherein determining the response of the test material to the gaseous mixture includes determining a degree of oxidation of the test material resulting from being exposed to the gaseous mixture.

9. The method of claim 8, wherein the method further comprises determining a thickness of the test material configured to withstand the degree of oxidation.

10. The method of claim 1, further comprising:
simulating flight conditions in a simulated flight environment using the gaseous mixture as a proxy for air in the flight conditions;
modulating flow characteristics of the gaseous mixture for a determined time period or queued time periods; and
monitoring oxidation of the test material during the determined time period or queued time periods.

11. The method of claim 1, further comprising:
identifying an oxygen gas (O₂) concentration in the gaseous mixture;
measuring oxidation of the test material at different oxygen gas (O₂) concentrations by modifying the gaseous mixture to include the different oxygen gas (O₂) concentrations;
generating an oxidation model of the test material based on the oxidation behavior at the different oxygen gas (O₂) concentrations; and
estimating oxidation behavior of the test material for potential deployment conditions using the oxidation model.

12. The method of claim 1, wherein decomposing the nitrous includes:
conditioning liquid nitrous into gaseous nitrous in the decomposition chamber;
injecting heated nitrogen gas (N₂) into the decomposition chamber so as to mix with and/or modulate the gaseous nitrous, wherein the injection of the heated nitrogen gas (N₂) is regulated to obtain the temperature for decomposition of the gaseous nitrous;
heating the gaseous nitrous to the temperature for decomposition of the gaseous nitrous;
decomposing the gaseous nitrous into the gaseous mixture, the gaseous mixture including the nitrogen gas (N₂) and the oxygen gas (O₂);
restricting injection of the heated nitrogen gas into the decomposition chamber to increase a concentration of the oxygen gas (O₂) in the gaseous mixture; and
analyzing the test material in the test chamber based on the increased concentration of oxygen gas (O₂) in the gaseous mixture.

13. The method of claim 1, comprising:
injecting heated nitrogen gas (N₂) into the decomposition chamber for a first period of time;
restricting or terminating injection of the heated nitrogen gas (N₂) into the decomposition chamber for a second period of time after the first period of time;
optionally, reinjecting the heated nitrogen gas (N₂) into the decomposition chamber for a third period of time after the second period of time; and
determining oxidation of the test material in the test chamber during the second period of time and/or third period of time.

14. The method of claim 1, comprising heating the nitrous to at least the temperature for decomposition, the temperature for decomposition being at least about 900 degrees K.

15. The method of claim 1, wherein determining the response of the test material includes determining a degree of oxidation of the test material resulting from combustion heat flux and wherein the method further comprises:
generating an oxidation model of the test material based on the response of the test material; and
determining a predicted degree of oxidation resulting from combustion heat flux under predetermined conditions based on the oxidation model.

16. The method of claim 1, further comprising:
monitoring oxidation of the test material in the test chamber; and
modeling the oxidation of the test material for different concentrations of oxygen and nitrogen in the gaseous mixture.

17. The method of claim 1, wherein a pressure sensitive paint is applied to a test surface of the test material before exposing the test material to the gaseous mixture and the method further comprises;
measuring brightness of luminescence of the pressure sensitive paint in response to the test material being exposed to the gaseous mixture;
determining areas of high brightness to have low oxygen concentration or low oxygen pressure;
determining areas of low brightness to have high oxygen concentration or high oxygen pressure; and
mapping a continuous pressure field of the test surface based on the measured brightness.

18. The method of claim 17, wherein the gaseous mixture has a nitrogen concentration higher than ambient air and an oxygen concentration of less than 21% by volume or moles when the test material is exposed to the gaseous mixture.

19. A method of controlling nitrogen and oxygen content of synthetic air, comprising:
decomposing nitrous in a decomposition system by expanding liquid nitrous into gaseous nitrous in a decomposition chamber of the decomposition system, wherein the gaseous nitrous obtains a temperature for decomposition;
obtaining a gaseous mixture of oxygen gas (O₂), transient oxygen atoms (O), nitrogen gas (N₂), and/or transient nitrogen atoms (N) from the decomposition of the nitrous, wherein the oxygen atom to nitrogen atom ratio is about 1:2 (oxygen atoms/molar: nitrogen atoms/molar);
providing the gaseous mixture to a test chamber;
providing an additional nitrogen gas (N₂) into the test chamber to obtain a nitrogenated gaseous mixture, wherein the test chamber optionally receives an additional oxygen gas (O₂), wherein the nitrogenated gaseous mixture has a nitrogen content of either:
less than or about 77% nitrogen gas (N₂) and transient nitrogen atoms (N) by volume or moles and greater than 22% oxygen gas (O₂) and transient oxygen atoms (O) by volume or moles; or
greater than 80% nitrogen gas (N₂) and transient nitrogen atoms (N) by volume or moles and less than 21% oxygen gas (O₂) and transient oxygen atoms (O) by volume or moles;
exposing a test material to the nitrogenated gaseous mixture in the test chamber; and
determining a response of the test material to the nitrogenated gaseous mixture.

20. The method of claim 19, wherein a pressure sensitive paint is applied to a test surface of the test material before exposing the test material to the gaseous mixture and the method further comprises;
measuring brightness of luminescence of the pressure sensitive paint in response to the test material being exposed to the gaseous mixture;
determining areas of high brightness to have low oxygen concentration or low oxygen pressure;
determining areas of low brightness to have high oxygen concentration or high oxygen pressure; and
mapping a continuous pressure field of the test surface based on the measured brightness.

21. A method of controlling nitrogen and oxygen content of synthetic air, comprising:
decomposing nitrous in a decomposition system by conditioning liquid nitrous into gaseous nitrous in a decomposition chamber of the decomposition system, wherein the gaseous nitrous obtains a temperature for decomposition;
obtaining a gaseous mixture of oxygen gas (O₂), transient oxygen atoms (O), nitrogen gas (N₂), and/or transient nitrogen atoms (N) from the decomposition, wherein the oxygen atom to nitrogen atom ratio is about 1:2 (oxygen atoms/molar: nitrogen atoms/molar);
providing the gaseous mixture to a test chamber;
providing an additional nitrogen gas (N₂) into the test chamber to obtain a nitrogenated gaseous mixture, wherein the test chamber is devoid of receiving additional oxygen gas (O₂) input, wherein the nitrogenated gaseous mixture has a nitrogen content of either:
less than or about 77% nitrogen gas (N₂) and transient nitrogen atoms (N) by volume or moles and greater than 22% oxygen gas (O₂) and transient oxygen atoms (O) by volume or moles; or
greater than 80% nitrogen gas nitrogen gas (N₂) and transient nitrogen atoms (N) by volume or moles and less than 21% oxygen gas (O₂) and transient oxygen atoms (O) by volume or moles;
exposing the nitrogenated gaseous mixture to a test material in the test chamber; and
determining a response of the test material to the nitrogenated gaseous mixture.

22. The method of claim 21, wherein a pressure sensitive paint is applied to a test surface of the test material before exposing the test material to the gaseous mixture and the method further comprises;
measuring brightness of luminescence of the pressure sensitive paint in response to the test material being exposed to the gaseous mixture;
determining areas of high brightness to have low oxygen concentration or low oxygen pressure;
determining areas of low brightness to have high oxygen concentration or high oxygen pressure; and
mapping a continuous pressure field of the test surface based on the measured brightness.
